(19) United States
(12) Patent Application Publication
(10) Pub. No.: US 2009/0293872 A1
(43) Pub. Date: Dec. 3, 2009

(54) ANESTHETIC BREATHING APPARATUS AND INTERNAL CONTROL METHOD FOR SAID APPARATUS

(76) Inventor: Hans Bocke, Taby (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/474,538

(22) Filed: May 29, 2009

(30) Foreign Application Priority Data
May 30, 2008 (SE) SE0801291-6

(51) Int. Cl. A61M 16/01

(52) U.S. Cl. 128/203.14; 128/203.12

(57) ABSTRACT

An anesthetic breathing apparatus has a breathing circuit having an inspiratory gas output port and an expiratory gas input port, and a control unit that sets the anesthetic breathing apparatus selectively in a first mode of operation to provide inspiratory gas, fresh gas and/or breathing gas recirculated in said breathing circuit, via said inspiratory gas output port, enable recirculation into said breathing circuit and/or evacuation via the expiratory input port for manual or mechanical ventilation by said anesthetic breathing apparatus. The control unit is also able to set the anesthetic breathing apparatus selectively in a second mode of operation to provide a flow of fresh gas via a fresh gas output port to an external breathing system connected thereto, disable recirculation and/or evacuation via the expiratory input port. An exhaust of the breathing circuit is connected to a gas input port of the anesthetic breathing apparatus for gas scavenging via said anesthetic breathing apparatus.

### BACKGROUND OF THE INVENTION

1. Field of the Invention

This invention pertains in general to the field of anesthetic breathing apparatuses and control methods therefor. More particularly the invention relates to the controlled delivery of gas from an anesthetic breathing apparatus and optionally controlled evacuation via an anesthetic breathing apparatus, e.g. to and optionally from an external breathing circuit connectable to a patient.

2. Description of the Prior Art

Known anesthetic apparatuses comprise a separate, gas outlet port in addition to an inspiratory gas outlet port, a so called additional fresh gas outlet port. The inspiratory gas outlet port is conventionally configured to be connected to the patient via suitable inspiratory tubing and a Y-piece. The additional fresh gas outlet port is activatable by e.g. operating a latch or lever, thus bypassing a breathing circle and the inspiratory output port. An output of fresh gas is thus provided via the additional fresh gas output port. The output of fresh gas is usually provided at a flow rate chosen by the user of the anesthetic breathing apparatus.

In U.S. Pat. No. 5,398,675 an anesthetic breathing apparatus is disclosed including a patient circuit. A separate, additional fresh gas outlet 158 is disclosed to which fresh gas is provided via valves, selectable via a manual control knob 200. The control knob may also be set to automatic ventilation mode, wherein the additional fresh gas outlet is deactivated and the patient circuit activated, providing gas to a connected patient via an inspiratory gas port.

A commercially available anesthetic breathing apparatus having an additional fresh gas outlet port is for instance the KION™ system. In the Kion™ system the additional fresh gas outlet port is activated by a dedicated lever. When the additional fresh gas outlet port is activated, delivery of gas to a patient connected to the inspiratory gas port is deactivated, which is indicated by a control indicator on a control panel of the KION™ system.

The additional fresh gas output port is in general not directly connected to a patient. Usually the additional fresh gas outlet port is arranged for connecting an external breathing system, such as an open breathing system or an external breathing circuit.

The additional fresh gas port is usually used when there are reasons that an internal breathing circuit of the anesthetic breathing apparatus is not desired to be used. This may for instance be the case when the user wants to control patient ventilation in a different manner. For instance for small patients like children and neonates, there may be a desire to manually ventilate the patient with a small systematic volume.

External breathing systems comprise for instance so called Bains, Jackson R, or similar external breathing circuits. The external breathing circuits have their own exhaust for waste gas, which hitherto is not returned to the anesthetic breathing apparatus for handling.

Moreover, open breathing systems generally may have difficulties handling the expiratory gas flow from the patient. It is desired that the gas expired from the patient is not released to the surrounding environment in order to avoid exposure to anesthetic agents.

Operating the above mentioned latch or lever, may be made unintentionally, thus bypassing the breathing circle. This may lead to reduced patient safety.

It may also be desired to provide a anesthetic breathing apparatus having reduced manufacturing cost, e.g. due to less parts than conventional anesthetic breathing apparatuses.

Thus, there is a need for an improved anesthetic breathing apparatus. The anesthetic breathing apparatus is desired to be more user friendly and to have improved patient safety, and a minimized possibility of connecting a patient in an undesired manner to the anesthetic breathing apparatus. In addition the anesthetic breathing apparatus may provide an improved handling of expired patient gas.

Hence, an improved anesthetic breathing apparatus would be advantageous and in particular allowing for increased flexibility, cost-effectiveness, patient safety and user friendliness would be advantageous.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing an anesthetic breathing apparatus, an internal control method for an anesthetic breathing apparatus, and a computer program.

In some embodiments, a flow of fresh gas is provided via a fresh gas output port to an external breathing system connected thereto. Recirculation and/or evacuation via an expiratory input port of the anesthetic breathing apparatus is disabled, and an exhaust of an external breathing system is connected to a gas input port of the anesthetic breathing apparatus for gas scavenging via the anesthetic breathing apparatus.

The conventionally existing additional fresh gas outlet port is omitted, not present, deactivated, or not used for supply of a flow of fresh gas in some embodiments of the present anesthetic breathing apparatus. Instead, the anesthetic breathing apparatus is internally controlled such that an existing inspiratory gas port is providing an auxiliary fresh gas flow, e.g. to external systems including external breathing systems. The inspiratory gas output port thus becomes multifunctional.

In this manner the anesthetic breathing apparatus contains less parts and has reduced cost of manufacture. In addition, unintentional activation of a fresh gas flow when a patient is connected is avoided.

According to one aspect of the invention, an anesthetic breathing apparatus is provided.

According to another aspect of the invention, an internal control method for an anesthetic breathing apparatus is provided.

According to a further aspect of the invention, a computer program for processing by a computer is provided.

In some embodiments, an anesthetic breathing apparatus or system has a breathing circuit having an inspiratory gas output port and an expiratory gas input port. The anesthetic breathing apparatus further has a control unit that is adapted to set the anesthetic breathing apparatus selectively in a first mode of operation to provide inspiratory gas, fresh gas and/or breathing gas recirculated in the breathing circuit, via the inspiratory gas output port; enable recirculation into the breathing circuit and/or evacuation via the expiratory input port for manual or mechanical ventilation by the anesthetic breathing apparatus. In use of the anesthetic breathing system in the first mode of operation, a patient is connected to the inspiratory gas output port and the expiratory gas input port.

Further, the control unit is adapted to set the anesthetic breathing apparatus selectively in a second mode of operation to provide a flow of fresh gas via a fresh gas output port of the system. In some embodiments, the output port for the fresh gas is the inspiratory gas output port. In some embodiments, a separate output port for the fresh gas is provided, wherein the inspiratory gas output port is disabled for output of fresh gas. In the second mode of operation, an external breathing system is connected to the output port for the fresh gas, and recirculation and/or evacuation via the expiratory input port is disabled. In use of the anesthetic breathing system in the second mode of operation, a patient is connected to the external breathing system.

In the anesthetic breathing system, an exhaust of the external breathing circuit is connected to a gas input port of the anesthetic breathing apparatus for gas scavenging, wherein the gas input port is different from the expiratory input port. In embodiments, the gas input port is a test port in a third mode of operation of the anesthetic apparatus, and wherein the control unit is adapted to activate the test port for the scavenging in the second mode of operation.

In some embodiments, a flow of the fresh gas in the second mode of operation is solely provided via the multifunctional inspiratory outlet port for external use, e.g. the external breathing system.

In some embodiments, a selection of the first or second mode of operation is provided automatically by recognition of a patient or external breathing circuit connected to the multifunctional inspiratory outlet port. The recognition may be based on a coded connection identification, such as based on barcode, RFID, electronic or mechanical coding, wherein a selection of the second mode of operation optionally is activated upon confirmation from a user of the anesthetic breathing apparatus.

In some embodiments, the flow of fresh gas is adjustable with regard to concentration of at least one specific gas component of the flow of fresh gas, such as O2; mixture of the gas components; a concentration of at least one anesthetic agent comprised in the fresh gas; and/or a total flow rate of the flow of fresh gas.

In some embodiments, the anesthetic breathing apparatus comprises a pressure measurement device arranged to measure a circuit pressure level of the flow of fresh gas at the multifunctional inspiratory outlet port. Thus, in some embodiments, the pressure in the external breathing circuit, when connected to the fresh gas output port, is monitorable or monitored by the anesthetic breathing apparatus.

In some embodiments, the control unit is adapted to deactivate from adjustment operating parameters of the anesthetic breathing apparatus related to circuit gas pressure levels or respiratory patterns, including Adjustable Pressure Limit (APL), Positive End Expiratory Pressure (PEEP), Respiratory Rate (RR), and Tidal Volume (TV); when in the second mode of operation.

In some embodiments, the control unit is adapted to only allow entering of the second mode of operation from a defined starting point including standby operation mode or manual ventilation operation mode of the anesthetic breathing apparatus.

In some embodiments, the control unit is adapted to only allow leaving the second mode of operation to the defined starting point.

In embodiments, a method of internally controlling an anesthetic breathing apparatus is provided. The method comprises selectively setting the anesthetic breathing apparatus in a mode of operation, for providing a flow of fresh gas via an inspiratory gas output port for connection of an external breathing system thereto, and disabling recirculation and/or evacuation via an expiratory input port of the anesthetic breathing apparatus. The method further comprises connecting an exhaust of an external breathing system to a gas input port of said anesthetic breathing apparatus for gas scavenging via said anesthetic breathing apparatus.

Further embodiments of the invention are defined in the dependent claims, wherein features for the second and subsequent aspects of the invention are as for the first aspect mutatis mutandis.

Some embodiments of the invention provide for a more simple anesthetic breathing apparatus.

Some embodiments of the invention provide for improved delivery of fresh gas to external systems from an anesthetic breathing apparatus.

Some embodiments of the invention provide for a less expensive anesthetic breathing apparatus and manufacturing thereof due to less parts than conventional anesthetic breathing apparatuses. Some embodiments of the invention provide for the omission of a dedicated sub-system for fresh gas delivery during a manual ventilation mode.

Some embodiments of the invention also provide for a controlled and monitorable delivery of a flow of fresh gas of desired composition and at a monitored circuit pressure to an external breathing circuit.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

FIG. 1 is a schematic drawing of a portion of an anesthetic breathing apparatus for conventional connection to a patient.

FIG. 2 is a schematic drawing of an anesthetic breathing apparatus for connection to an external breathing system, comprising a mode control unit 50 for providing an Auxiliary Fresh Gas Flow (AFGF) mode of operation.

FIG. 3 is a schematic drawing of a portion of an anesthetic as of FIG. 2 with an additional gas evacuation port.

FIG. 4 is a schematic illustration of an embodiment of an internal control method for an anesthetic breathing apparatus.

FIG. 5 is a schematic illustration of an embodiment of a computer program for internal control of an anesthetic breathing apparatus.

FIG. 6 is a schematic drawing of a portion of an alternative anesthetic breathing apparatus for connection to a patient and an alternative AFGF mode.

FIG. 7 is a schematic drawing of a portion of an alternative anesthetic breathing apparatus of an embodiment for an AFGF mode.

FIG. 8 is a schematic illustration of an anesthetic breathing system implementing an AFGF mode.

### DESCRIPTION OF THE PREFERRED EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The following description focuses on an embodiment of the present invention applicable to a particular anesthetic breathing apparatus or anesthetic breathing system. However, it will be appreciated that the invention is not limited to this particular apparatus or anesthetic breathing system may be applied to many other anesthetic breathing apparatuses or anesthetic breathing systems. For instance gas delivery portions of the anesthetic breathing apparatuses or anesthetic breathing systems may be constructed differently, anesthesia reflectors or adsorbers, CO2 absorbers, etc., may be included to breathing circles or not; gas delivery valves and mixing of gas components or delivery of anesthetic agents may be constructed differently.

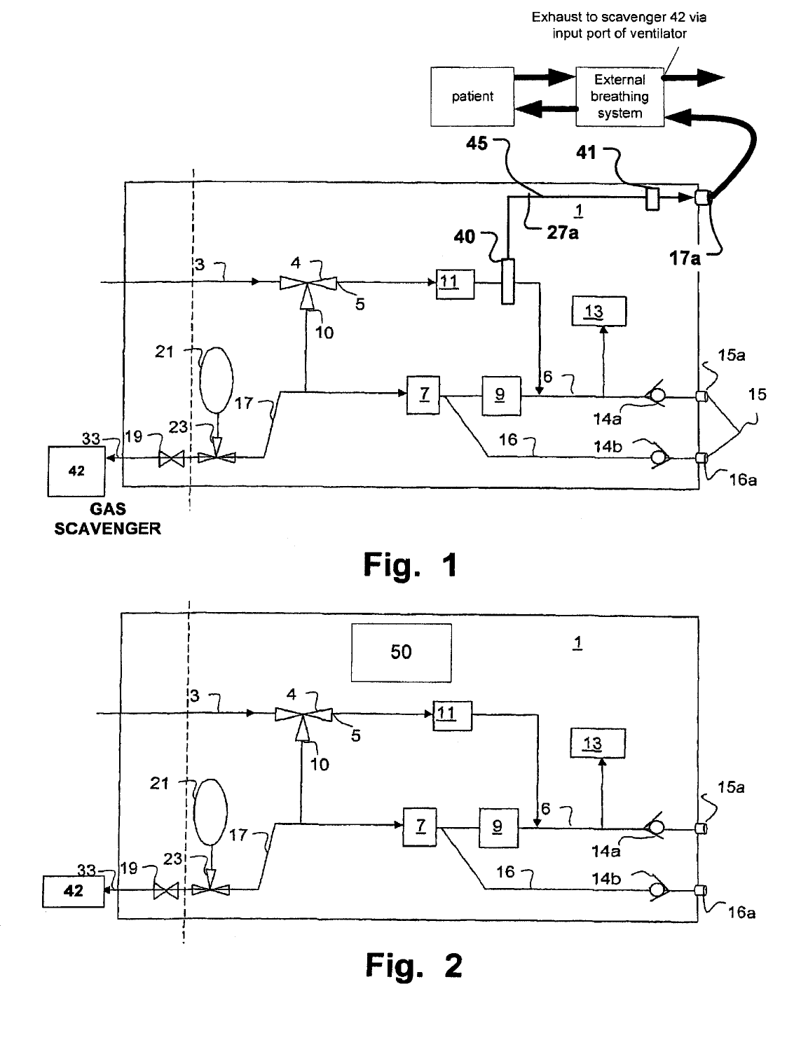

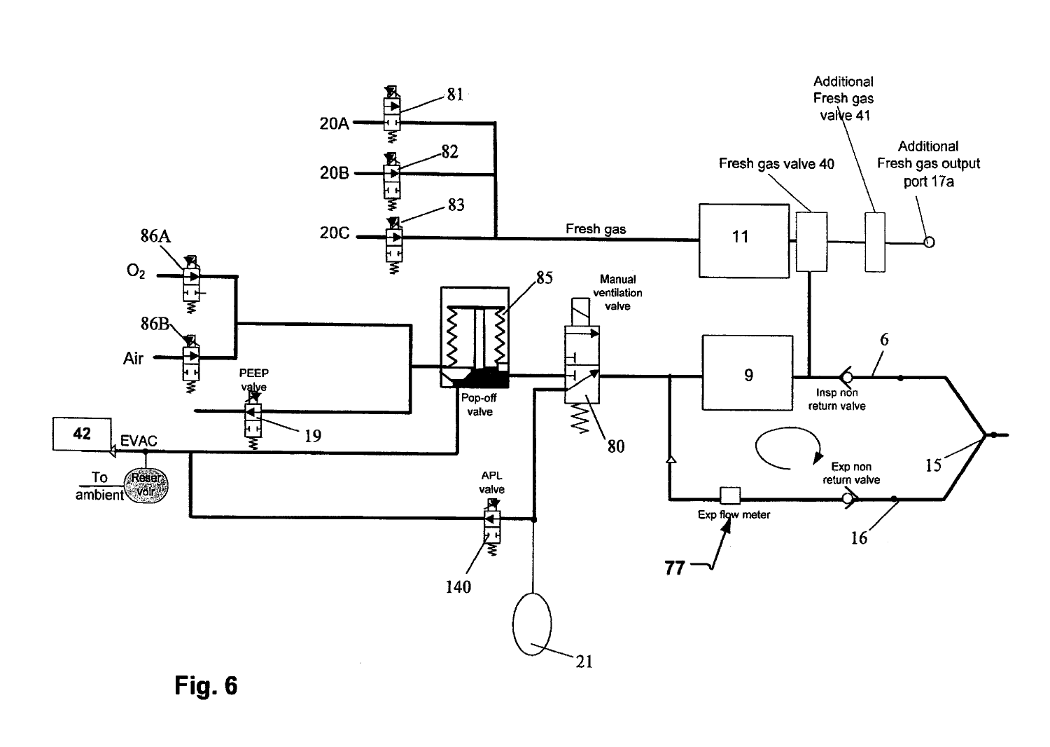

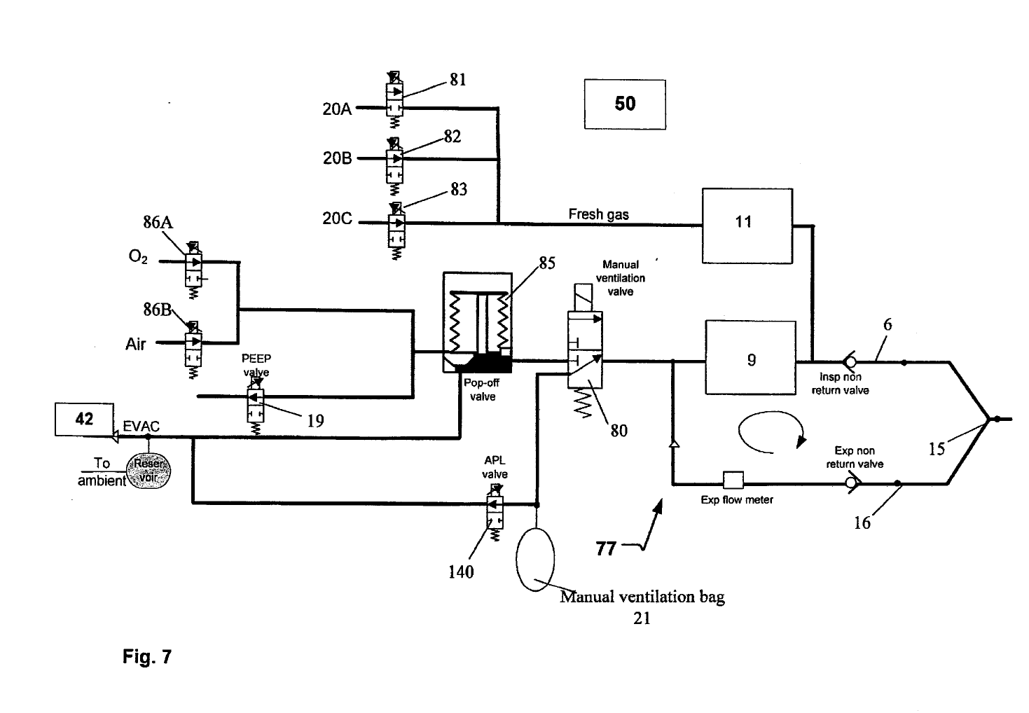

For instance, in WO 2007/0071756 alternative anesthetic breathing apparatuses are disclosed that are suitable to be modified according to the present invention, see in particular FIGS. 1, and 5-7 thereof and the related description. WO 2007/071756 is incorporated herein by reference in its entirety for all purposes. FIG. 7 of WO 2007/0071756 with an additional fresh gas output port is shown in FIG. 6 of the present application.

A software controlled Adjustable Pressure Limit (APL) valve is also disclosed in WO 2007/071756, which is incorporated herein by reference in its entirety for all purposes. Software controlled APL valves may be controlled suitably by a control unit as described below, with reference to adjusting various operation modes of an anesthetic breathing apparatus.

FIG. 1 is a schematic drawing of a portion of an anesthetic breathing apparatus for conventional connection to a patient. A breathing circuit 1 comprises an input 3 from a ventilator (not shown) to an adjustable selection valve, or shunt valve 4. Through a first outlet 10 of the shunt valve 4, the main part of the breathing gas is provided to a common line 17. Said common line may comprise an anesthesia reflector 7, either a carbon filter reflector or a volume reflector as is known in the art. See for instance U.S. Pat. No. 5,471,979 or U.S. Pat. No. 4,989,597, which are incorporated herein by reference in their entirety for all purposes. The common line is branched to an inspiration line 6 carrying breathing air to the patient and an expiration line 16 carrying expired air from the patient. A CO2 absorber 9 is connected in series with the anesthesia reflector 7 in the inspiration line 6 between the anesthesia reflector 7 and the patient.

Through a second outlet 5 of the shunt valve 4 a portion of the breathing gas may be provided to a vaporizer 11. A vaporized anesthetic agent is provided from the vaporizer 11 to the inspiration line 6 between the absorbing filter and the patient.

Delivery of fresh gas to external systems is provided by a separate fresh gas line 45, fresh gas selection valve 40, fresh gas activation lever 41 and additional fresh gas port 17a. Upon activation of the fresh gas activation lever 41, the fresh gas selection valve 40 is activated to provide a flow of fresh gas from shunt valve 4 via the outlet 5 and the vaporizer 11. In addition, the first outlet 10 is deactivated. As described in the background section above, an external breathing system may be connected to the additional fresh gas port 17a.

Between the vaporizer 11 and the patient, a gas analyzer 13 is connected to the inspiration line 6 to monitor and control the composition of the gas, such as the concentration of anesthetic agent.

When in use for automated, mechanical ventilation by the anesthetic breathing system, a Y-piece (schematically shown as 15) is used for connection to a patient, as is common in the art. The Y piece is connected, by means of tubes to the inspiration line 6 through a non-return valve 14a and to the expiration line 16 through a non-return valve 14b. The expiration line 16 and the inspiration line 6 are joined between the anesthesia reflector 7 and the CO2 absorber 9.

The point of the inspiration line to which the inspiratory tube towards the Y piece 15 is connected is referred to as the inspiration gas port 15a, and the point of the expiration line to which the expiratory tube from the Y piece is connected is referred to as the expiration gas port 16a.

From the anesthesia reflector 7 exhaled air is evacuated through the common line 17 to an evacuation line 33 for connection to a central evacuation/scavenging system 42, or other exhaust gas retaining means (not shown). A positive end expiratory pressure (PEEP) valve 19 is connected in the common line near the evacuation line 33 for controlling the end expiratory pressure in the breathing circuit. Near the PEEP valve 19 on the breathing system side a manual ventilation bag 21 is connected through a manual ventilation valve 23. The evacuation line 33 is connected to a scavenging system (not shown) downstream of the PEEP valve 19. The PEEP function relates to the machine working in controlled mechanical mode or support mode. In these modes the manual bag is disabled.

In manual mode, with the manual bag enabled, the PEEP valve 19 is disabled and a separate APL valve (e.g. APL valve 140 shown in FIG. 6) controls the pressure in the breathing circuit. Optional the valve is software controlled and has the function of an APL valve in manual mode.

Technically, the PEEP valve is part of the ventilator, while all other components shown in FIG. 1 are included in the anesthesia breathing circuit. The dashed line in FIG. 1 indicates the border between the ventilator on the left and the anesthesia breathing circuit on the right of the dashed line. The breathing circuit to the right of the dashed border may be integrated in a removable box or cassette, e.g. of plastics, defining the gas channels. Such a box or cassette is designed to be easily removed for cleaning and service purposes.

In an alternative setup, not shown, the vaporizer block 11 may be connected in the inspiration line 6 between the CO2 absorber 9 and the non-return valve 14a. The breathing gas from the shunt valve 4 is then introduced between the absorber and the vaporizer, or directly into the vaporizer.

There are a number of components that are self evident in an anesthesia circuit, but not shown, such as pressure and flow meters. These units provide data such as patient inspiratory and expiratory pressures and flows to a control unit.

The use of the apparatus in FIG. 1 is apparent to a person skilled in the art and need not be discussed in detail.

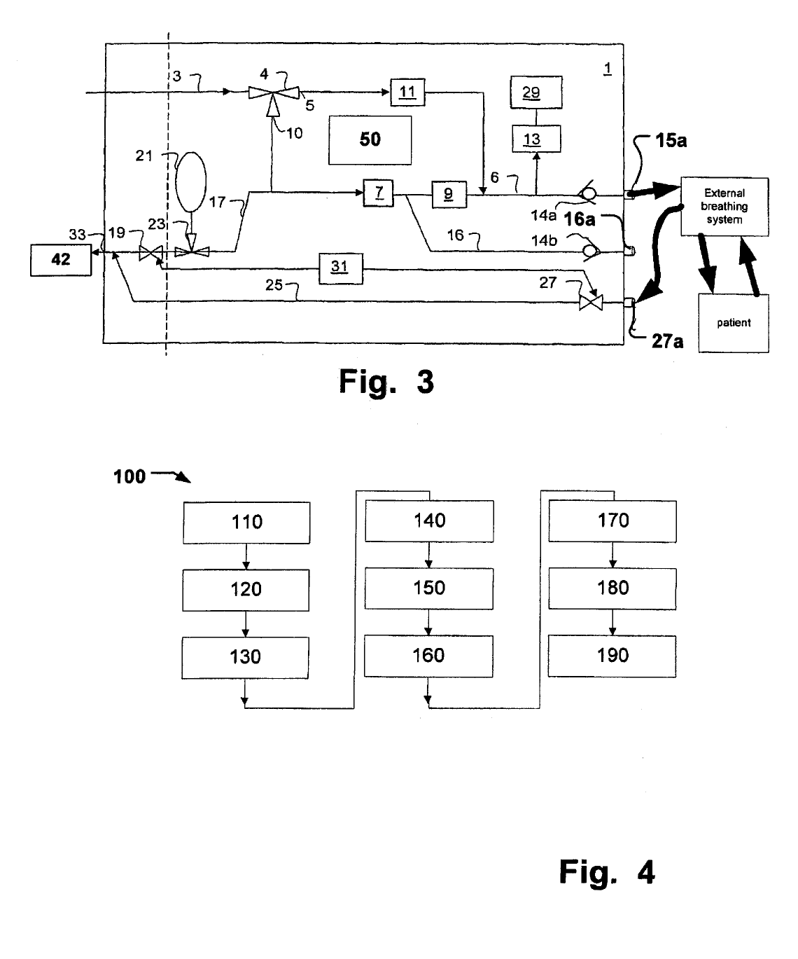

FIGS. 2 and 3 show essentially the same anesthetic apparatus as FIG. 1, but with modifications according to embodiments of the invention to enable delivery of fresh gas to an external breathing system (not shown) and scavenging of exhaust gas from the external breathing system. As can be seen, the apparatus as shown in FIGS. 2 and 3 have no separate fresh gas line 45, fresh gas selection valve 40, fresh gas activation lever 41 and additional fresh gas port 17a. These components, and similar components for additional fresh gas delivery ports, can be omitted and are not necessary in embodiments, as will be clear from the description below.

As those skilled in the art will realize, the modifications made to the anesthetic breathing apparatus for enabling delivery of fresh gas to an external breathing system (not shown) and scavenging of exhaust gas from the external breathing system, as described in connection with FIG. 2 and FIG. 3, may be made to any known anesthetic breathing apparatus, for example, also to anesthetic breathing apparatus not comprising a anesthesia reflector, or an anesthetic breathing apparatus in which the vaporizer is serially connected with the anesthesia reflector and the absorber in the inspiration line. Also, of course, the ventilator (not shown) may provide a desired mixture of breathing gases.

Other modifications may comprise leaving, and perhaps deactivating, existing additional fresh gas delivery ports, and performing modifications in order to provide auxiliary fresh gas delivery via an existing gas port of a known anesthetic breathing apparatus.

Auxiliary Fresh Gas Flow (AFGF) Mode of Operation In an embodiment, the additional fresh gas outlet functionality is provided by providing a fresh gas flow to an inspiratory outlet on a breathing circuit of an anesthetic breathing apparatus, see FIG. 2.

An anesthetic breathing apparatus is provided, in which the number of gas output ports is minimized. This improvement in some embodiments further comprises a reduction of number of electrical or mechanical selector switches. An existing inspiratory output port is provided, which is configured to supply a desired, monitorable fresh gas flow, e.g. to an external breathing circuit.

To this end, the anesthetic breathing apparatus is provided with a specific Auxiliary Fresh Gas Flow (AFGF) mode of operation.

The number of gas output ports is thus minimized, as an additional fresh gas output port is omitted. Thus manufacturing cost of the anesthetic breathing apparatus is minimized. Also, the anesthetic breathing apparatus is more user friendly and is provided with increased patient safety as an unintentional bypass of a breathing circuit by unintentionally activating an additional fresh gas port is avoided.

In the AFGF mode the anesthetic breathing apparatus provides a fresh gas flow via the inspiratory output port. Thus, the inspiratory output port is extended to a multi function gas output port, both connectable to a patient via the inspiratory limb of a Y-piece (during automated ventilation or manual ventilation via the anesthetic breathing apparatus) and as a pure fresh gas output to an external gas management system, such as an external breathing circuit (in AFGF mode). In AFGF mode, the expiratory tube from the Y piece is not connected to the expiration gas port 16a, as expiration is controlled by the external gas management system, such as the external breathing circuit.

In AFGF mode the anesthetic breathing apparatus is arranged to deliver a unique fresh gas flow. Other parameters that usually are provided by an anesthetic breathing apparatus and related to gas pressure levels or respiratory patterns are deactivated, including Adjustable Pressure Limit (APL), Positive End Expiratory Pressure (PEEP), Respiratory Rate (RR), and Tidal Volume (TV). These parameters are inactive during AFGF mode and may not be adjusted as long as the AFGF mode is active. A manual breathing bag 21 is deactivated during AFGF mode.

However, fresh gas flow related parameters may be suitably adjustable by a user during AFGF mode, including concentration of specific gas components, such as O2; mixture of gas components; anesthetic agent concentration; total flow rate. The AFGF parameters comprise e.g. a Requested Constant Fresh Gas Flow; a Requested Gas Concentration Parameters, e.g. for O2; a Requested Gas Mix; a requested Anesthetic Gas Concentration, e.g. a concentration or effective dose of halogenated fluorocarbon anesthetic agents, such as Isoflurane, Desflurane, Sevoflurane, etc. A maximum pressure or a patient release pressure in AFGF mode may be a fixed value in dependence of patient category (adult, child, neonate), or variable and adjustable by the user.

The total fresh gas flow from the additional fresh gas outlet function provided at the inspiratory outlet port may be adjustable, e.g. in a range from 0 to 20 l/min, such as in predetermined fixed values in the range, for instance [0, 0.3, 0.4, . . . , 20] l/min. Alternatively, or in addition, variable or continuous adjustment of the flow of fresh gas is provided.

The anesthetic breathing system allows the user to adjust the Auxiliary Fresh Gas Flow parameters while the system is in Auxiliary Fresh Gas Flow Mode.

By using usual monitoring units provided in an anesthetic breathing apparatus or system, the gas delivered via the inspiratory output port is monitorable. For instance fresh gas parameters like gas composition or concentration of fresh gas components, circuit pressure, etc. may be monitored.

Measured parameters in AFGF mode may comprise circuit pressure, e.g. measured by a pressure sensor or pressure measurement device arranged to measure circuit pressure between the inspiratory check valve 14a and the inspiratory gas outlet port 15a. An external sampling point, such as at Y-piece 15, may provide further measurement data. In addition, or alternatively, an external pressure sensor or pressure measurement device may be provided.

The AFGF Data is the AFGF measured data and may be displayed both numerically and as a function of time.

In case an external gas delivery system, such as an external breathing circuit, is connected to the inspiratory outlet port, these fresh gas parameters may be provided for further processing. Further processing may include calculations; storage; display or visualization of values, graphical curves, like trending, etc., e.g. on a user interface like a graphical monitor.

This monitoring allows for improved patient safety as e.g. a maximum pressure may be controlled in AFGF mode. This was hitherto not possible. Furthermore, visualization of e.g. pressure curves is provideable. The circuit pressure in the external breathing system may be visualized in curve form, as numerical values, and/or trended.

### AFGF Operation Mode

AFGF operation mode is started from a defined starting point, e.g. the anesthetic breathing system is in standby mode or manual ventilation mode. For security reasons, the anesthetic breathing system may only allow the user to enter the Auxiliary Fresh Gas Flow Mode from Standby mode. In order to improve patient safety, the AFGF mode may suitable only be activated in operation of the anesthetic breathing apparatus where it is ensured that a patient is not directly connected to the inspiratory gas port. This may for instance be done at the starting point, e.g. in standby operation or manual ventilation operation. A specific warning may be provided on a display to make the user of the anesthetic breathing apparatus aware of activating AFGF mode.

The user disconnects the normal patient tubing ((at least) from the Inspiratory Outlet) and connects whatever (breathing) system he wants to the Inspiratory Outlet port 15a.

When trying to activate AFGF mode in automatic ventilation mode of the anesthetic breathing system, this is not allowed by the anesthetic breathing apparatus, or the control unit 50 thereof. For instance, if the operator tries to activate AFGF by pushing an AFGF activation button or similar during automatic ventilation, AFGF mode will not be activated due to patient security reasons. Via a graphical user interface (GUI) of the anesthetic breathing system, the anesthetic breathing system may inform the user that automatic ventilation needs to be exited when wanting to switch to AFGF mode. Unintentional activation of AFGF mode during automatic ventilation is avoided. The user has to take a suitable action, e.g. set the ventilation mode to manual ventilation operation, or put the anesthetic breathing apparatus into standby operation, or the defined starting point. A protective point or lock may has to be surpassed by the user in order to improve patient safety.

AFGF is only activatable when the anesthetic breathing apparatus or system is in the defined starting point.

An example of AFGF operation mode is given below, with reference to FIG. 4:

Normal Flow:

100. The user chooses to activate the AFGF mode;

110. When at the above mentioned starting point, and entering of AFGF mode is allowed, the anesthetic breathing system starts AFGF mode and closes the APL valve and manual ventilation valve 23 and then applies a set of the aforementioned AFGF parameters, Fresh Gas and Anesthetic Gas Supply, wherein all action is controlled by control unit 50.

120. The anesthetic breathing system continuously shows the AFGF Data.

130. The anesthetic breathing system continuously supervises the AFGF Alarms.

140. Repeatedly:

150. the user adjusts AFGF parameters such as the fresh gas flow and gas concentrations, and

160. the anesthetic breathing system adapts the gas flow and mix accordingly;

170. The user exits AFGF mode, e.g. a soft button “Turn off AFGF” is activated. Alternatively, or in addition, a hardware button or switch is activated. Exiting AFGF mode may further be confirmed, e.g. in a user dialogue via a GUI.

180. The anesthetic breathing system goes back to the starting point and as a consequence stops the Fresh Gas Flow and turns the vaporizers off.

190. The system opens the APL and Manual Ventilation Bag Valves.

When AFGF mode is turned off, the anesthetic breathing system returns to the starting point.

Alternatively, AFGF mode may be terminated when switching to the starting point.

Further, the anesthetic breathing system may only allow the user to leave the Auxiliary Fresh Gas Flow Mode by going to Standby mode.

In case the anesthetic breathing system detects a high continuous patient pressure during the AFGF mode, the system may raise an alarm, turn of the fresh gas flow, closes the vaporizer valves and open the Safety Valve. In case the anesthetic breathing system detects a temporary high patient pressure, or peak pressure, the system may raise an alarm, and open the Safety Valve.

The anesthetic breathing may stay in AFGF mode. Alternatively, for increased security, e.g. when a patient is connected, alarms may be raised, and AFGF mode halted, paused, or aborted back to the starting point.

Additional Gas Scavenging of External Breathing Circuit In some embodiments the exhaust of the external breathing circuit may be connected to a gas input port of the anesthetic breathing apparatus for gas scavenging. Such an embodiment is for instance illustrated with reference to FIG. 3.

For instance, the exhaust of the external breathing circuit, may be coupled to a test port 27a, when present in the anesthetic breathing system. The disclosure of WO2008/000299, of the same applicant as the present application, and which is hereby is incorporated by reference in its entirety for all purposes, comprises such a test port. The test port is internally connected to a gas scavenging output port of the anesthetic breathing apparatus. As described in WO2008/000299 the test port is originally devised for use in pre use check or a system check out, which in operation for instance is connected to a hospital gas evacuation system for handling of waste gas.

An additional evacuation line 25 has an evacuation valve 27. A control unit 31 that executes a computer program could be provided for controlling the evacuation valve and the PEEP valve in a third mode of operation, e.g. during a pre-use check. The evacuation valve 27 is controlled in such a way that it is closed during the phases of mechanical ventilation and open for AFGF for connection to an exhaust of an external breathing system. The control unit 31 is shown as a separate unit but is suitably integrated in the control unit 50 of the anesthetic breathing system. For simplicity, the control system is shown connected only to the PEEP valve 19 and the evacuation valve 27. Also, the control unit 31 may be arranged to receive an input signal from a gas analyzer, indicating a concentration of anesthetic agent or CO2 in the exhaust gas flow. This input signal may thus be used by the control unit 31 to determine when the external breathing system is connected to port 27a.

Thus, when the external breathing circuit is exhausting gas comprising an anesthetic agent, it is effectively avoided that anesthetic agent is released to the surrounding environment, which is advantageous. A further evacuation system, which otherwise would be needed for the external breathing system to avoid possible contamination of the surrounding environment with volatile anesthetic is not needed. The anesthetic system having an AFGF port and an exhaust port, thus provides a more cost-effective solution.

In a specific embodiment a System Checkout plug for an Anesthetic Gas Scavenging System is used in an AFGF mode for controlled evacuation of gas returned from the breathing system that is externally connected to the anesthetic breathing apparatus.

The port 27a may be provided with embodiments of multi functional AFGF ports, such as described with reference to FIG. 1 or 6, or dedicated AFGF ports, such as described with reference to FIG. 2, 3, 7 or 8.

FIG. 6 is a schematic drawing of a portion of an alternative anesthetic breathing apparatus for connection to a patient, having an alternative AFGF mode. The implementations of FIGS. 6, 7 and 8 include a “bag in bottle” 85 to drive the breathing gas in a circle system 77. This bag in bottle 85 is provided with a, so called, pop-off valve 89, releasing excess gas from the breathing circuit to an evacuation system. Driving gas in the mechanical ventilation system is provided to the outside side of the bag in bottle 85. An expiratory valve PEEP valve 19 controls the pressure of the driving gas, e.g. by regulating a restriction in the evacuating line, as illustrated. Thereby the flow of the driving gas through the expiratory valve 19 to evacuation (EVAC) is regulated, and the pressure in the breathing circle is controlled by the pressure of the drive gas by means of the bag in bottle 85. Gas sources (O2 and air) are connected via supply valves 86A, 86B, respectively, and further the bag in bottle 85. In this way the O2 and air sources 86A, 86B provide driving gas, during mechanical ventilation, to the bag driving the breathing gas inside the bag and which driving gas pressure is adjusted by, means of controlling, the expiratory valve 19. The manual ventilation bag 21 is connected to the evacuation line, including the expiratory valve 19, via a junction 90. Between the bag 21 and the junction 90 a manual ventilation valve 48 may be provided for selecting manual ventilation. The junction 90 connects the manual ventilation bag 21 to the expiration branch with the expiratory valve and to the breathing circle via a selection valve 80 and a common inspiration and expiration line. Fresh gas is supplied to the breathing circuit 77 from gas source 20A, 20B, 20C via valves 81, 82, 83, respectively.

FIG. 7 is a schematic drawing of a portion of an alternative anesthetic breathing apparatus of an embodiment implementing an AFGF mode, wherein the AFGF mode is software controlled by unit 50.

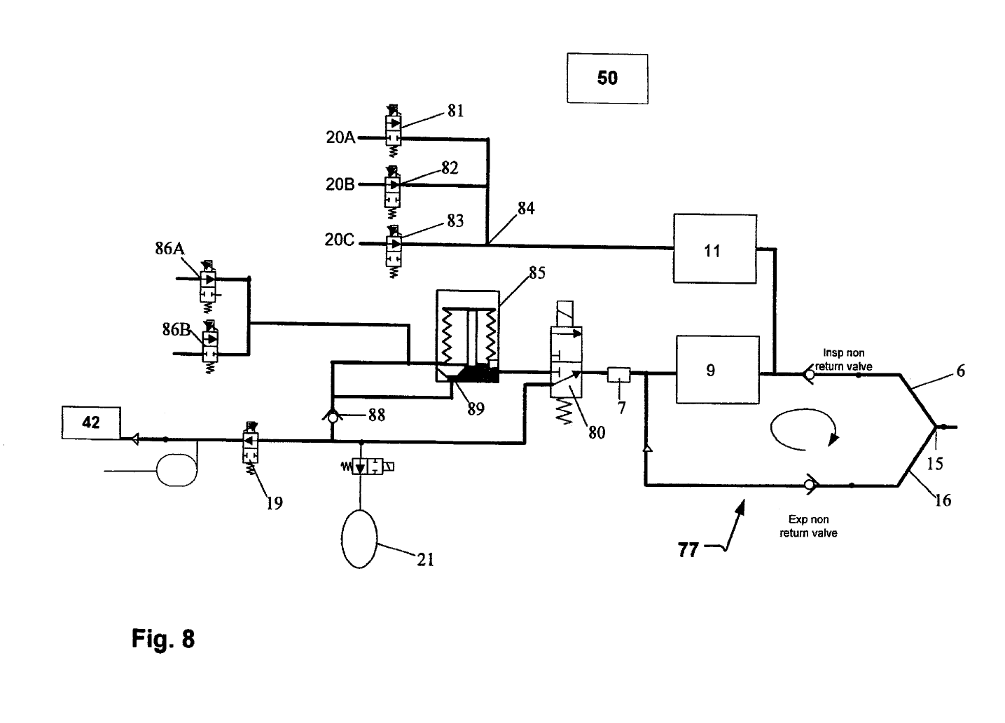

FIG. 8 is a schematic illustration of an anesthetic breathing system as described in PCT/EP2007/056880 of the same applicant as the present application, filed 06 Jul. 2007, which hereby is incorporated by reference in its entirety for all purposes.

AFGF mode may in the anesthetic breathing systems of FIGS. 6 to 8 be selected according to above, by deactivating mechanical ventilation and activating AFGF mode for delivery of a desired and monitored fresh gas supply to an external breathing system. The patient is disconnected from the inspiratory and expiratory connections of the anesthetic breathing system and connected to the external breathing system, as described above, for AFGF mode. The exhaust from the external breathing system may for AFGF mode be connected to a separate port, such as port 27a (not shown in FIGS. 6 to 8).

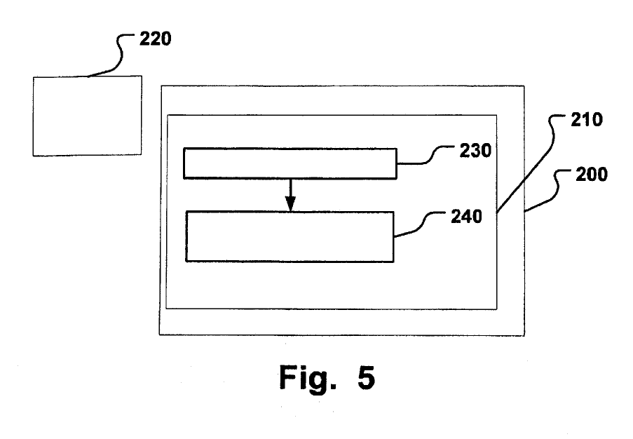

A computer program 210 for performing the above described method is shown in FIG. 5, stored on a computer readable medium 200 and for execution by a computing device 220, such as a central processing unit (CPU). The computer program 210 comprises code segments 230, 240 for performing the method as shown in FIG. 4.

The present invention has been described above with reference to specific embodiments. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims.

I claim as my invention:

1. An anesthetic breathing apparatus comprising:
a breathing circuit having an inspiratory gas output port and an expiratory gas input port and an exhaust:
a control unit configured to set said breathing circuit selectively in a first mode of operation to provide inspiratory gas, fresh gas and/or breathing gas recirculated in said breathing circuit, via said inspiratory gas output port, and to enable recirculation into said breathing circuit and/or evacuation via said expiratory input port for manual or mechanical ventilation by said anesthetic breathing apparatus;
said control unit being configured to set said anesthetic breathing apparatus selectively in a second mode of operation to provide a flow of fresh gas via a fresh gas output port to an external breathing system connected thereto, and to disable recirculation and/or evacuation via said expiratory input port; and
a gas scavenger fluidly connected to the exhaust of said breathing circuit via a separate gas input port of the anesthetic breathing apparatus for gas scavenging via said anesthetic breathing apparatus.

2. The anesthetic breathing apparatus according to claim 1, wherein said gas input port is a test port in a third mode of operation of said anesthetic breathing apparatus, and wherein said control unit is adapted to activate said gas input port for said scavenging in said second mode of operation.

3. The anesthetic breathing apparatus according to claim 1, wherein said fresh gas output port is said inspiratory outlet port, and wherein said inspiratory outlet port is provided with multifunctional properties.

4. The anesthetic breathing apparatus according to claim 3, wherein a flow of said fresh gas in said second mode of operation is solely provided via said multifunctional inspiratory outlet port for external use by said breathing system.

5. The anesthetic breathing apparatus according to claim 3, wherein said control unit is configured select said first or second mode of operation automatically by recognition of a patient or external breathing circuit connected to said multifunctional inspiratory outlet port, wherein said recognition is based on a coded connection identification, selected from the group consisting of barcode, RFID, electronic or mechanical coding, and to select said second mode of operation optionally upon confirmation from a user of said anesthetic breathing apparatus.

6. The anesthetic breathing apparatus according to claim 1, wherein said flow of fresh gas is adjustable with regard to concentration of at least one specific gas component of said flow of fresh gas, selected from the group consisting of O₂ a mixture of said gas components, a concentration of at least one anesthetic agent comprised in said fresh gas, and a total flow rate of said flow of fresh gas.

7. The anesthetic breathing apparatus according to claim 6, wherein said control unit is configured to deactivate from adjustment operating parameters of said anesthetic breathing apparatus related to circuit gas pressure levels or respiratory patterns, including Adjustable Pressure Limit (APL), Positive End Expiratory Pressure (PEEP), Respiratory Rate (RR), and Tidal Volume (TV).

8. The anesthetic breathing apparatus according to claim 1, wherein said control unit is configured to only allow entering of said second mode of operation from a defined starting point including standby operation mode or manual ventilation operation mode of said anesthetic breathing apparatus.

9. The anesthetic breathing apparatus according to claim 8, wherein said control unit is configured to only allow leaving said second mode of operation to said defined starting point.

10. The anesthetic breathing apparatus according to claim 1, comprising a pressure measurement device that measures a circuit pressure level of said flow of fresh gas at said fresh gas outlet port.

11. The anesthetic breathing apparatus according to claim 1, comprising an evacuation valve connected to said gas input port, said evacuation valve being controlled by said control unit to be closed during said first mode of operation and open for said second mode of operation.

12. A method of internally controlling an anesthetic breathing apparatus, said method comprising:
selectively setting said anesthetic breathing apparatus in a mode of operation, comprising providing a flow of fresh gas via a fresh gas output port to an external breathing system connected thereto, disabling recirculation and/or evacuation via an expiratory input port of said anesthetic breathing apparatus; and
connecting an exhaust of an external breathing system to a gas input port of said anesthetic breathing apparatus for gas scavenging via said anesthetic breathing apparatus.

13. A computer-readable medium loadable into a control unit for internally controlling an anesthetic breathing apparatus, comprising programming code causing said control unit to selectively set said anesthetic breathing apparatus in a mode of operation, for:
providing a flow of fresh gas via a fresh gas output port to an external breathing system connected thereto;
disabling recirculation and/or evacuation via an expiratory input port of said anesthetic breathing apparatus; and
wherein an exhaust of an external breathing system is connected to a gas input port of said anesthetic breathing apparatus for gas scavenging via said anesthetic breathing apparatus.
